(19) United States
(12) Patent Application Publication
(10) Pub. No.: US 2009/0076590 A1
(43) Pub. Date: Mar. 19, 2009

(54) ENDOPROSTHESES WITH STRUT PATTERN HAVING MULTIPLE STRESS RELIEVERS

(75) Inventor: Karl P. Keating, Co. Galway (IE)

Correspondence Address: FULWIDER PATTON, LLP (ABBOTT), 6060 CENTER DRIVE, 10TH FLOOR, LOS ANGELES, CA, 90045, US

(73) Assignee: ABBOTT LABORATORIES, Abbott Park, IL (US)

(21) Appl. No.: 11/854,721

(22) Filed: Sep. 13, 2007

(51) Int. Cl. A61F 2/82

(52) U.S. Cl. 623/1.16

(57) ABSTRACT

An endoprostheses for implanting in a body lumen, such as a coronary artery, peripheral artery, or other body lumen can be formed with a structure, referred to as a stress reliever, designed to distribute and reduce the amount of strain which can act on the movable struts of the stent. Stress relievers can be disposed at a strut junction where one end of a strut is attached to the end of an adjacent strut. The positioning and shape of the stress reliever help to distribute the amount of strain that would otherwise be exerted on the movable struts as the struts move relative to each other. As a result, there is less possibility that the struts will fracture at the strut junction when the struts move from a collapsed position to an expanded position.

### BACKGROUND OF THE INVENTION

The invention relates generally to vascular repair devices, and in particular to endoprostheses, more commonly referred to as intravascular stents, which are adapted to be implanted into a patient's body lumen, such as a blood vessel or artery, to maintain the patency thereof. Stents are particularly useful in the treatment of atherosclerotic stenosis in arteries and blood vessels. More particularly, the present invention is directed to an intravascular stent having a pattern or configuration that reduces the amount of stress and strains experienced by the struts of the stent during deployment and when the struts are subjected to physiological deformations that can cause a high degree of fracture and fatigue to the stent.

Stents are generally tubular-shaped devices which function to hold open a segment of a blood vessel or other body lumen such as a coronary or peripheral artery. They also are suitable for use to support and hold back a dissected arterial lining that can occlude the fluid passageway. At present, there are numerous commercial stents being marketed throughout the world. While some of these stents are flexible and have the appropriate radial rigidity needed to hold open a vessel or artery, there typically is a tradeoff between flexibility and radial strength.

Prior art stents typically fall into two general categories of construction. The first type of stent is expandable upon application of a controlled force, often through the inflation of the balloon portion of a dilatation catheter which, upon inflation of the balloon or other expansion means, expands the compressed stent to a larger diameter to be left in place within the artery at the target site. The second type of stent is a self-expanding stent formed from shape memory metals or super-elastic nickel titanium (NiTi) alloys, which will automatically expand from a compressed state when the stent is advanced out of the distal end of the delivery catheter into the blood vessel. Such stents manufactured from expandable heat sensitive materials usually allow for phase transformations of the material to occur, resulting in the expansion and contraction of the stent.

Stents can be implanted in the coronary arteries along with peripheral arteries, for example, the renal arteries, the carotid arteries and long arterial segments in the leg, all of which are susceptible to arteriosclerosis. Generally, balloon-expandable stents have been implanted in the coronary arteries since the coronary arteries are generally not vulnerable to bending and compression forces that can distort the stent structure. Typically, balloon-expandable stents are made from a stainless steel or cobalt-chromium alloy, multi-layer materials or other similar biocompatible materials. Peripheral vessels, on the other hand, are usually more prone to natural bending and compressive forces which can easily bend and distort the implanted stent, causing it to fracture. Due to its material make up, a balloon-expandable stent usually is not implanted in peripheral arteries that are subject to repetitive bending and compressive forces since these forces will likely deform and/or fracture the balloon-expandable stent. For this reason, self-expanding stents are usually implanted in peripheral vessels since the self-expanding properties of the stent allows it to spring back to shape even after being subjected to bending or compressive forces.

Stent placement in long segments of the peripheral arteries, such as the ilio-femoral-popliteal artery, can be challenging to the stent manufacturer since there are regions in these peripheral arteries where bending and compressive forces are so constant and repetitive that even a self-expanding stent is subjected to possible deformation caused by fatigue and fracturing. Other regions of peripheral arteries are subject to compressive forces which can prevent the stent from possibly spring back to its open, expanded configuration which can lead to stent fracture as well. For example, it has been shown that the ilio-femoral-popliteal segment undergoes non-pulsatile deformations which will, in turn, act on any stent implanted in this arterial segment. These deformations have been identified as being axial, torsional and/or bending and specific segments of the superficial femoral artery have been associated with specific non-pulsatile deformations. These deformations can impinge on the stent's ability to maintain these arteries in a fully opened position and can result in deformation and fracturing of the often intricate strut patterns which form the stent structure.

In many procedures which utilize stents to maintain the patency of the patient's body lumen, the size of the body lumen can be quite small which prevents the use of some commercial stents which have profiles which are entirely too large to reach the small vessel. Many of these distal lesions are located deep within the tortuous vasculature of the patient which requires the stent to not only have a small profile, but also high flexibility to be advanced into these regions. As a result, the stent must be sufficiently flexible along its longitudinal axis, yet be configured to expand radially to provide sufficient strength and stability to maintain the patency of the body lumen.

What has been needed and heretofore unavailable is a stent structure having a high degree of flexibility so that it can be advanced through tortuous passageways and can be radially expanded in a body segment, and yet possesses sufficient mechanical strength to hold open the body lumen or artery to provide adequate vessel wall coverage while reducing the amount of strain exerted on the struts of the stent during stent expansion and when the stent is subjected to fracture and fatigue strains resulting from physiological deformations. The present invention satisfies these and other needs.

### SUMMARY OF THE INVENTION

The present invention is directed to an intravascular stent having a strut pattern or configuration that distributes and reduces the amount of strain exerted on the expandable struts of the stent during stent deployment and also helps to distribute and reduce the amount of strain which can be exerted on the stent struts when the stent is implanted in certain body vessels that produce physiological deformations that can act on the implanted stent. The stent is highly flexible along its longitudinal axis to facilitate delivery through tortuous body lumens, but is stiff and stable enough radially in its expanded condition to maintain the patency of a body lumen, such as an artery, when the stent is implanted therein.

A stent made in accordance with the present invention can be formed with a structure, referred to as a stress reliever, which is designed to distribute and reduce the amount of strain which can act on the movable struts of the stent. Stress relievers can be disposed at a strut junction where one end of a strut is attached to the end of an adjacent strut. The positioning and shape of the stress reliever help to distribute the amount of strain that would otherwise be exerted on the movable struts as the struts move relative to each other. As a result, there is less possibility that the struts will fracture at the strut junction when the struts move from a collapsed position to an expanded position. These stress relievers also help to distribute and reduce the amount of strain placed on the struts at the strut junction once the expanded stent is implanted in the body vessel. The stress relievers mitigate the amount of fatigue strain placed on the stent which can be caused by physiological deformations associated with particular body vessels, such as the peripheral vessels in the leg, which are highly susceptible to bending and compressive forces that will act on the implanted stent and cause fatigue and fracture of the often intricate strut patterns forming the stent structure.

In one aspect of the present invention, the stent includes a series of strut pairs which collectively form the expandable stent body. Each strut pair includes two adjacent struts which are connected together at a strut junction. At least one stress reliever is associated with each strut pair in order to distribute and reduce the amount of strain experienced at the strut junction. In one aspect of the invention, one or more stress relievers can be placed at the strut junction between adjacent struts.

In one aspect of the present invention, two or more stress relievers can be placed between adjacent struts to help the struts bend when moving into the expanded or deployed position. In this regard, as adjacent struts rotate or move towards each other, the stress relievers bend towards each other to allow the connected ends of the struts to more easily rotate or bend. This structure distributes the strain at the strut junction to reduce the strain placed on the connected ends of the struts. In this configuration, the stress relievers remain in compression when the struts are placed in the expanded position.

In a similar fashion, some adjacent struts will be moving away from each other as the stent body moves into the expanded position. The stress relievers placed between two adjacent struts which move away from each other behave differently than the stress relievers described above since the stress reliever will remain in tension, not compression, as the stent body assumes the expanded position. However, in accordance with the present invention, the structure and placement of stress reliever will reduce the amount of strain acting at the connected ends of the struts.

The number of adjacent struts which can be commonly connected together can be two or more, depending, of course, on the desired strut pattern. In this regard, one or more stress relievers can be placed at the strut junction in between adjacent struts. In this fashion, as the struts move from a collapsed position to an expanded position, the stress relievers will act to distribute and reduce the amount of strain exerted on the ends of the struts that are connected at the strut junction. In one particular aspect of the present invention, four adjacent struts form a strut group which serves to create the stent body. Each of the four struts can be connected at a single strut junction which includes one or more stress relievers disposed between each of the adjacent struts. This strut group will form a X-shaped structure once placed in the expanded position. Again, the stress relievers will help to prevent strut fracture at the strut junction by distributing and reducing the amount of deployment strain exerted at the connected ends of the struts.

In one particular aspect of the present invention, the strut reliever is in the form of an upright circular peak (an upright projection) and includes the formation of a notched radius directly adjacent to each connected end of strut pair at a given strut junction. The placement of a notched radius adjacent to the connected ends of each strut helps to prevent strut fracture along the ends of the struts. In use, the stress reliever creates a “web” of the stent material which bridges the connected ends of the stent struts at a strut junction to reduce and more evenly distribute strains which may act on the struts at the strut junction.

The stent may be formed from a tube by laser cutting the pattern of struts and stress relievers in the tube. The stent also may be formed by laser cutting a flat metal sheet in the pattern of the elongate struts and links, and then rolling the pattern into the shape of the tubular stent and providing a longitudinal weld to form the stent. As used throughout the present application, the term adjacent may be used to define directly adjacent or indirectly adjacent.

Other features and advantages of the present invention will become more apparent from the following detailed description of the invention when taken in conjunction with the accompanying exemplary drawings.

### BRIEF DESCRIPTION OF THE DRAWINGS

FIG. 1 is an elevational view, partially in section, of one particular embodiment of a stent made in accordance with the present invention mounted on a stent delivery catheter and positioned within an artery.

FIG. 2 is an elevational view, partially in section, similar to that shown in FIG. 1 wherein the stent is expanded within the artery, so that the stent contacts the arterial wall.

FIG. 3 is an elevational view, partially in section, showing the expanded stent implanted within the artery after withdrawal of the stent delivery catheter.

FIG. 4 is a plan view of a flattened stent of one embodiment of the invention which illustrates the pattern of struts and stress relievers when the stent is in an unexpanded position.

FIG. 5 is a plan view of the stent of FIG. 4 which shows the stent is the expanded position.

FIG. 6 is a perspective view of the stent depicted in FIG. 4.

FIG. 7 is an enlarged partial plan view depicting struts and stress relievers formed at a strut junction when the struts are in an unexpanded position.

FIG. 8 is an enlarged partial plan view depicting the struts, stress relievers and strut junction of FIG. 6 when the struts are placed in an expanded position.

### DETAILED DESCRIPTION OF THE PREFERRED EMBODIMENTS

The present invention stent improves on existing stents by providing a longitudinally flexible stent having uniquely designed stress relievers associated with the strut junctions of a stent which provides a high degree of fracture and fatigue resistance to the movable struts when the struts move to an expanded position or when subjected to physiological deformations associated with some body vessels. In addition to providing longitudinal flexibility, the stent of the present invention also provides radial rigidity and a high degree of scaffolding of a vessel wall, such as a peripheral artery.

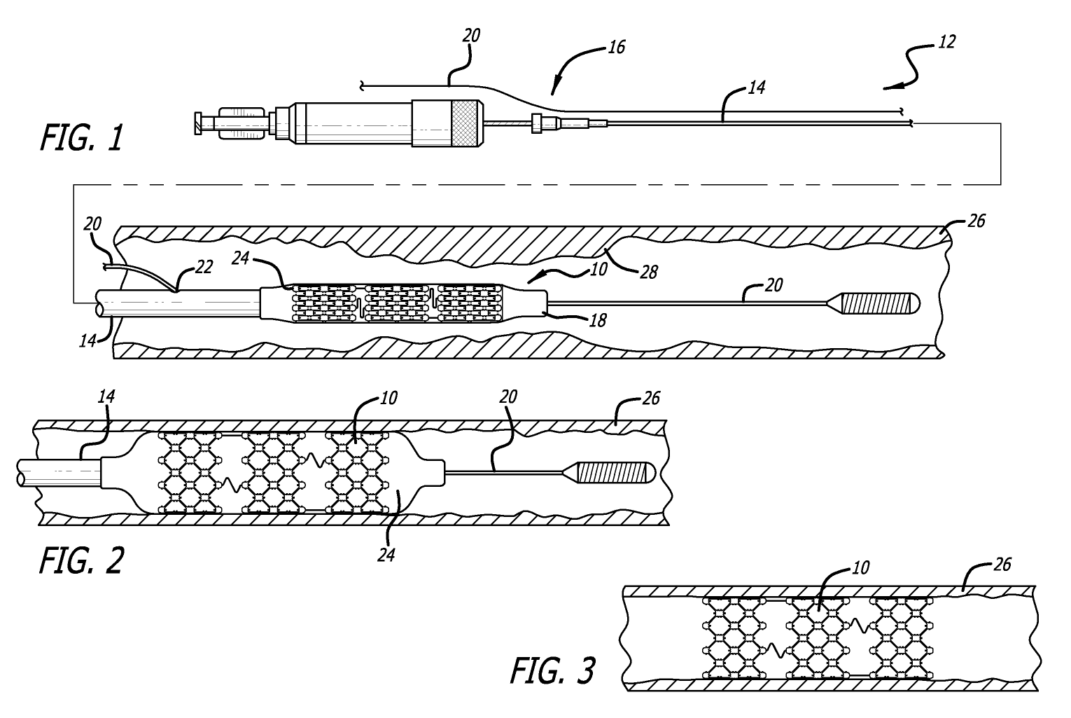

Turning to the drawings, FIGS. 1-3 depict a stent 10 made in accordance with the present invention as it is mounted on a conventional catheter assembly 12 used to deliver the stent 10 and implant it in a body lumen, such as a peripheral artery, a coronary artery or other vessel within the body. The catheter assembly includes a catheter shaft 14 which has a proximal end 16 and a distal end 18. The catheter assembly 12 is configured to advance through the patient's vascular system by advancing the catheter assembly 12 over a guide wire 20 using well known methods associated with over-the-wire or a well-known rapid-exchange catheter system, such as the one shown in FIG. 1.

Catheter assembly 12 as depicted in FIG. 1 is of the well known rapid exchange type which includes an RX port 22 where the guide wire 20 will exit the catheter. The distal end of the guide wire 20 exits the catheter distal end 18 so that the catheter advances along the guide wire 20 on a section of the catheter between the RX port 22 and the catheter distal end 18. As is known in the art, the guide wire lumen which receives the guide wire is sized for receiving various diameter guide wires to suit a particular application. The stent is mounted on the expandable member 24 (balloon) and is crimped tightly thereon so that the stent and expandable member present a low profile diameter for delivery through the arteries.

As shown in FIG. 1, a partial cross-section of an artery 26 is shown with a small amount of plaque 28 that has been previously treated by an angioplasty or other repair procedure. Stent 10 is used to repair a diseased or damaged arterial wall which may include plaque 28 as shown in FIGS. 1-3, or a dissection, or a flap of the arterial wall which is sometimes found in the peripheral and coronary arteries and other vessels.

In a typical procedure to implant stent 10, the guide wire 18 is advanced through the patient's vascular system by well known methods so that the distal end of the guide wire is advanced past the plaque or diseased area 26. Prior to implanting the stent, the cardiologist may wish to perform an angioplasty procedure or other procedure (i.e., atherectomy) in order to open the vessel and remodel the diseased area. Thereafter, the stent delivery catheter assembly 12 is advanced over the guide wire so that the stent is positioned in the target area. The expandable member or balloon 24 is inflated by well known means so that it expands radially outwardly and in turn expands the stent radially outwardly until the stent is apposed to the vessel wall. The expandable member is then deflated and the catheter withdrawn from the patient's vascular system. The guide wire typically is left in the lumen for post-dilatation procedures, if any, and subsequently is withdrawn from the patient's vascular system. As depicted in FIGS. 2 and 3, the balloon is fully inflated with the prior art stent expanded and pressed against the vessel wall, and in FIG. 3, the implanted stent remains in the vessel after the balloon has been deflated and the catheter assembly and guide wire have been withdrawn from the patient.

The stent 10 serves to hold open the artery 26 after the catheter is withdrawn, as illustrated by FIG. 3. Due to the formation of the stent 10 in the shape of an elongate tubular member, the undulating components of the stent 10 are relatively flat in transverse cross section, so that when the stent is expanded, it is pressed into the wall of the artery and as a result does not interfere with the blood flow through the artery. The stent 10 is pressed into the wall of the artery and may eventually be covered with endothelial cell growth which further minimizes blood flow interference. The undulating portion of the stent provides good tacking characteristics to prevent stent movement within the artery. Furthermore, the closely spaced connecting links found at regular intervals along the length of the stent provide uniform support for the wall of the artery, as illustrated in FIG. 3.

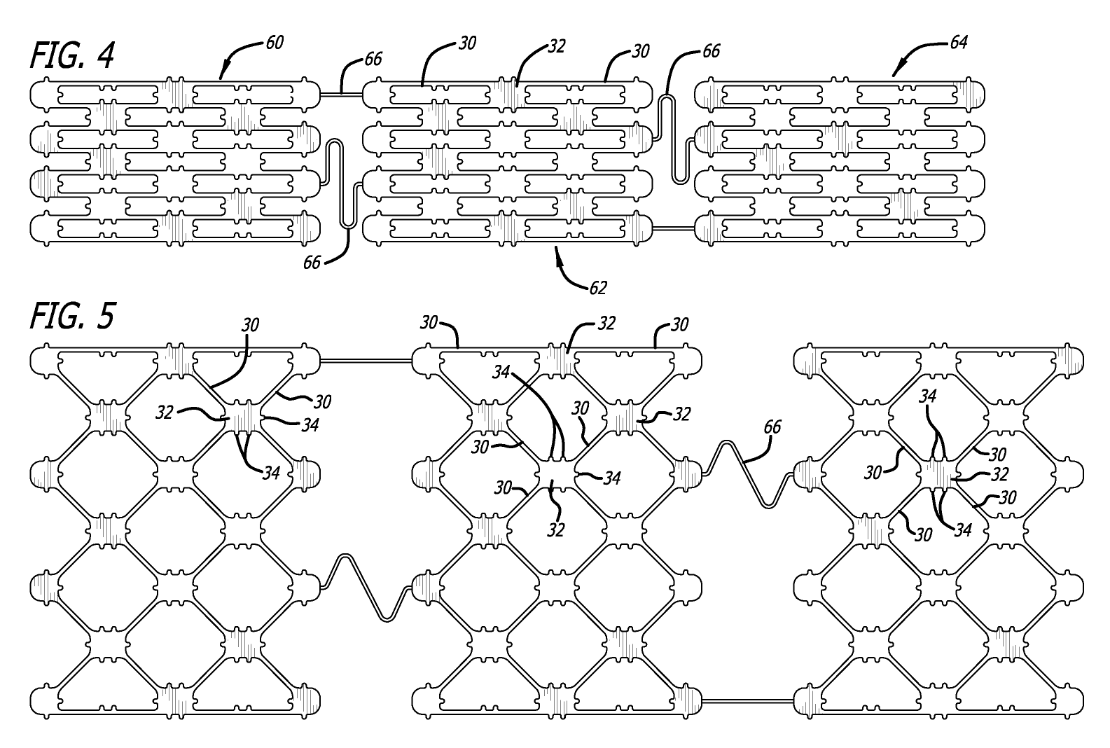

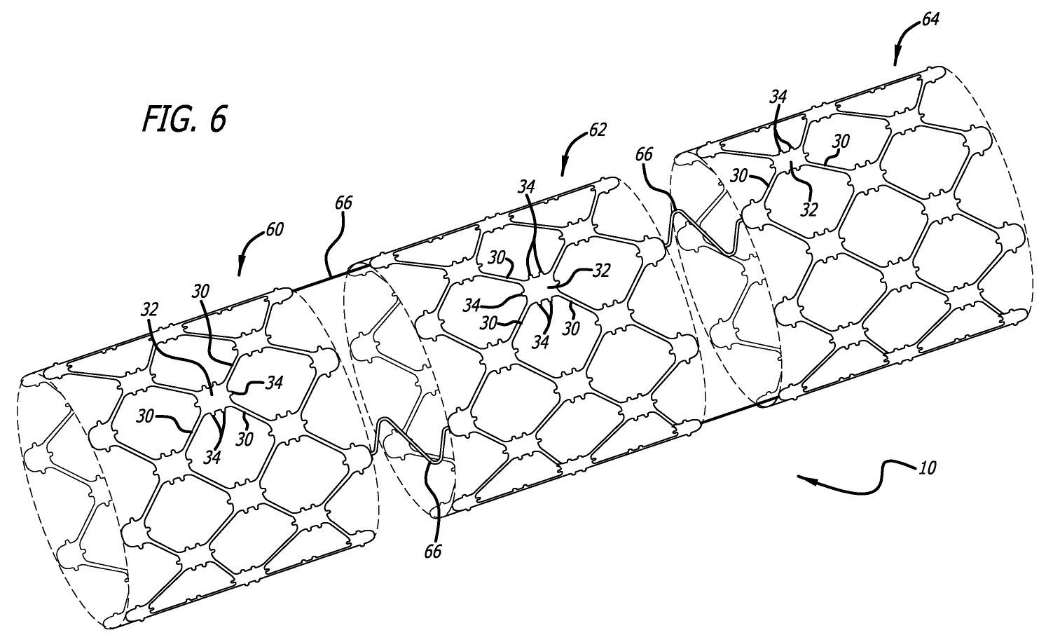

In keeping with the present invention, FIGS. 4-6 depict stent 10 in various configurations. The stent embodiments and patterns as disclosed herein are illustrative and by way of example only. The pattern can vary and still incorporate the stress relievers associated with the present invention. Referring to FIGS. 4 and 5, for example, stent 10 is shown in a flattened condition so that the pattern can be clearly viewed, even though the stent is in a cylindrical form in use, such as shown in FIG. 6. The stent is typically formed from a tubular member, however, it can be formed from a flat sheet and rolled into a cylindrical configuration.

Referring specifically to FIGS. 4-8, a stent made in accordance with the present invention includes a series of struts 30 which are connected to each other at a strut junction 32. The strut junctions 32 are designed to connect two or more struts 30 to allow the series of struts 30 and strut junctions to form the stent body forms in generally tubular or cylindrical shape when placed in the expanded position. See FIG. 6 which shows a perspective view which shows several stent segments which are connected together to form a unitary stent body.

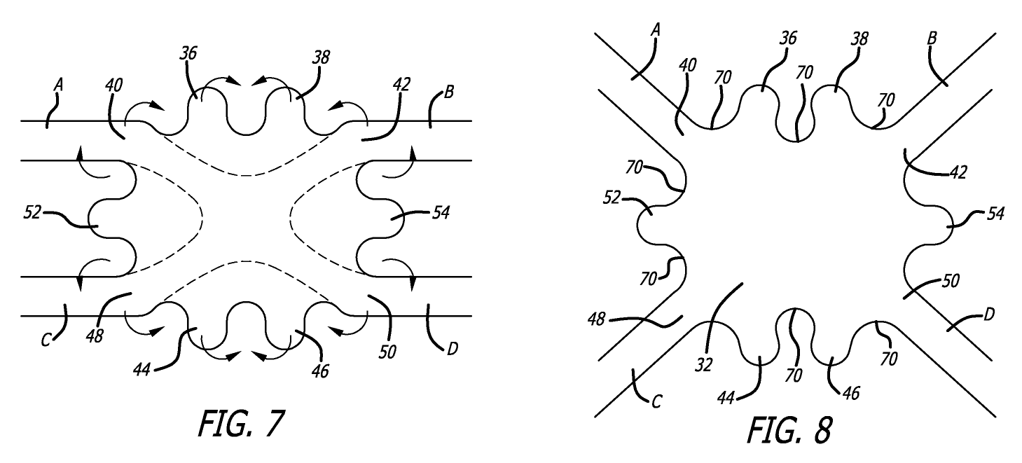

Each strut junction 32 includes one or more stress relievers 34 which are disposed at the circumference of the strut junction 32 and are formed between adjacent struts. These stress relieves 34 help to distribute and reduce the amount of stress that the struts 30 undergo when they move from a collapsed or unexpanded position, such as shown in FIG. 4, to an expanded position as is shown in FIGS. 5 and 6. FIG. 7 also shows an individual strut junction 32 in which four individual struts A, B, C and D are connected. In FIG. 7, the four struts A, B, C and D are shown in the collapsed position, as is further depicted in FIG. 4. Numerous arrows shown in FIG. 7 indicate the particular direction of motion in which the four struts A, B, C and D move when being radially expanded from the expanded position shown in FIGS. 8 and 6 into the expanded position shown in FIGS. 5 and 8.

As can be seen in FIGS. 7 and 8, struts A and B are designed to move toward each other as these struts move from the collapsed position to the expanded position. Likewise, struts C and D move towards each other as indicated by the arrows in FIG. 7. FIG. 8 shows the particular position that all of the struts A, B, C and D will assume once fully expanded. While struts A and B move towards each other, as does struts C and D, the opposite is true with regard to strut pairs A and C and B and D. As can be seen in FIGS. 7 and 8, arrows depict the direction of motion between struts A and C as these struts move into the radially expanded position shown in FIG. 8. The arrows show that struts A and C move away from each other as these struts move into the expanded position. The same is true for strut pairs B and D as these two struts will move away from each other once placed in the expanded position. Each of the strut pairs have at least one stress reliever 34 associated with the strut pair in order to distribute and reduce the amount of strain experienced by the strut pair as the struts move from a collapsed to an expanded position. For example, a pair of stress relievers 36 and 38 are shown placed between strut pair A and B. In use, these stress relievers 36 and 38 will move toward each other, as is shown by the arrows in FIG. 7, when strut pair A and B move towards each other when moving into the expanded position. FIG. 8 depicts these stress relievers 36 and 38 closer to each other than is shown in FIG. 7. In this regard, the stress relievers 36 and 38 act to distribute the stresses and strains experienced at the strut junction 32 to help reduce the possibility that the connected ends 40 and 42 of struts A and B can possible fracture as the struts move between the collapsed and expanded positions. These stress relievers 36 and 38 remain in compression as the strut pair A and B move into the expanded position.

Similarly, struts C and D include a pair of stress relievers 44 and 46 which also move towards each other as the strut pair C and D are radially expanded, as is shown by the arrows in FIG. 7. These particular stress relievers 44 and 46 act in the same fashion as does the stress relievers 36 and 38 described above. These stress relievers 44 and 46 help to prevent fractures which could result to the connected ends 48 and 50 of struts C and D as they move from the collapsed to the expanded position.

A stress reliever 52 is also placed between the connected ends 40 and 48 of strut pair A and C as is shown in FIGS. 7 and 8. This particular stress reliever 52 unlike the previously described stress relievers 36, 38, 46 and 48 remains in tension as strut pair A and C move away from each other when the are placed into the expanded position as is shown in FIG. 8. Similarly, a stress reliever 54 is placed between strut pair B and D. This particular stress reliever 54, like stress reliever 52, remains in tension as strut pair B and D move away from each other. In this regard, these stress relievers 36, 38, 46, 48, 52 and 54 act to distribute and reduce the amount of strain that would otherwise be exerted at the connected ends 40, 42, 48 and 50 of the struts A, B, C and D, respectively. These stress relievers also help to reduce the amount strain experienced at the connected ends of the struts when the stent is implanted in a body vessel which experiences physiological deformations which are in turn exerted on the implanted stent. Again, these stress relievers help to prevent fatigue fractures which may occur at the strut junction 32.

The number of stress relievers between the struts can be varied depending upon the material properties and how much strain is exerted between the struts. For example, if the material has high compressive strength and low tensile strength, then more stress relievers would be placed in areas that experience high compressive strains. This may be balanced for deployment strains and in use fatigue stains. It should be appreciated that although the present invention shows only two stress relievers placed between struts A and B, that additional or even less stress relievers could be utilized depending upon the amount of strain that is exerted between the struts and the material properties of the stent. Similarly, more than one stress reliever could be placed between struts A and C which again depends upon the amount of stress experienced in this area, along with the material properties of the stent.

The present invention is shown with a number of pairs of struts which are connected at their ends to a strut junction which includes the use of stress relievers in accordance with the present invention. In some instances, two ore more struts can be attached at the strut junction. For example, as can be seen in the particular embodiment of the strut pattern shown in FIGS. 4-8, the stent pattern includes strut pairs attached to a particular strut junction along with four struts connected at a single strut junction. It would be appreciated by those skilled in the art that two or more struts could be attached to a single strut junction, again depending upon the particular strut pattern which is desired to be created. The number of stress relievers place at the strut junction between strut pairs will depend, for example, upon the direction of motion that the strut pair takes relative to each other during expansion, along with the material properties of the stent.

FIGS. 4-6 show one in particular embodiment of a stent in which two or more stent segments 60, 62 and 64 are formed and connected together to form a single stent body. In this regard, each stent segment is connected to an adjacent stent segment utilizing connecting links 66 which cooperatively creates a composite stent body. It should appreciated by those skilled in the art that although three stent segments 60, 62 and 64 are shown in this particular embodiment, a single segment could be made and used as a unitary stent. The length of the stent segment could be sized as needed for a given application.

The stress relievers of the present invention can be used with numerous stent patterns in which two or more struts are connected at a strut junction. Additionally, stress relievers do not have to be placed at each and every strut junction in any particular stent pattern. The number used and the particular placement of stress relievers can vary depending on the stent pattern and particular strength of the material used to form the stent.

Referring specifically again to FIGS. 7-8, the particular size and shape of each stress reliever will be explained. FIG. 7 shows dotted lines which depict the strut junction 32 in the absence of the various stress relievers used in accordance with this invention. As can be seen in FIG. 7, the ends of the struts A, B, C and D would ordinarily be quite thin and fragile in the absence of the stress relievers. As a result, as the struts move relative to each other, or if there is strain applied to the connected ends 40, 42, 48 and 50 of the struts A, B, C and D, there is a distinct possibility that the ends of the struts can fracture, which is highly undesirable. The placement of each stress reliever between strut pairs again helps to dissipate and distribute the stresses which would otherwise act upon the ends of these struts.

As can be seen in FIGS. 7 and 8, each strut reliever is in the form of an upright circular peak (an upright projection) and includes the formation of a notched radius 70 directly adjacent to each connected end 40, 42, 48 and 50 of struts A, B, C and D. The placement of a notched radius adjacent to the connected ends of each strut helps to prevent strut fracture along the ends of the struts. It should appreciate by those skilled in the art that although the particular stress reliever is shown as a circular shaped, upright peak or projection, still other shapes could be utilized to attain the strain relieve at the connected ends of the struts.

In use, the stress reliever creates a “web” of the stent material which bridges the connected ends of the stent struts at a strut junction to reduce and more evenly distribute strains which may act on the struts at the strut junction. Normally, the stress reliever would have the same wall thickness, i.e., the measure of stent material from its inner surface to its outer surface, as the struts which form the stent, especially if the stent is laser cut from a tubular member. However, it is possible to reduce the wall thickness of the stress reliever if necessary. For example, the wall thickness of the stress reliever could be reduced by moving the laser beam over the stress reliever to remove a portion of the outside surface of the stress reliever during stent manufacturing. It should be appreciated by those skilled in the art that other methods for reducing the wall thickness of the stress reliever could also be utilized as well.

In accordance with the present invention, a stent and its stress relievers can be made from a self-expanding material or a balloon expandable material. A suitable composition of Nitinol used in the manufacture of a self-expanding stent of the present invention is approximately 55% nickel and 44.5% titanium (by weight) with trace amounts of other elements making up about 0.5% of the composition. It should be appreciated that other compositions of Nitinol can be utilized, such as a nickel-titanium-platinum alloy, to obtain the same features of a self-expanding stent made in accordance with the present invention.

The stent of the present invention can be laser cut from a tube of nickel titanium (Nitinol). All of the stent diameters can be cut with the same stent pattern, and the stent is expanded and heat treated to be stable at the desired final diameter. The heat treatment also controls the transformation temperature of the Nitinol such that the stent is superelastic at body temperature. The transformation temperature is at or below body temperature so that the stent will be superelastic at body temperature. The stent can be electro-polished to obtain a smooth finish with a thin layer of titanium oxide placed on the surface. The stent is usually implanted into the target vessel which is smaller than the stent diameter so that the stent applies a force to the vessel wall to keep it open.

The stent tubing of a stent made in accordance with the present invention may be made of suitable biocompatible material besides nickel-titanium (NiTi) alloys. In this case, the stent would be formed using known techniques for manufacturing balloon expandable stents as well. The tubing may be made, for example, a suitable biocompatible material such as stainless steel. The stainless steel tube may be alloy-type: 316L SS, Special Chemistry per ASTM F138-92 or ASTM F139-92 grade 2. The stent of the present invention also can be made from a metallic material or an alloy such as, but not limited to, cobalt chromium alloy (ELGILOY), MP35N, MP20N, ELASTINITE, tantalum, platinum-iridium alloy, gold, magnesium, or combinations thereof. MP35N and MP20N are trade names for alloys of cobalt, nickel, chromium and molybdenum available from Standard Press Steel Co., Jenkintown, Pa. MP35N consists of 35% nickel, 20% chromium, and 120% molybdenum. MP20N consists of 50% cobalt, 20% nickel, 20% chromium, and 20% molybdenum. Stents also can be made from bioabsorbable or biostable polymers.

One method of making the stent, however, is to cut a thin-walled tubular member, such as Nitinol tubing, and remove portions of the tubing in the desired pattern for the stent, leaving relatively untouched the portions of the metallic tubing which are to form the stent. The tubing can be cut in the desired pattern by means of a machine-controlled laser.

Generally, the tubing is put in a rotatable collet fixture of a machine-controlled apparatus for positioning the tubing relative to a laser. According to machine-encoded instructions, the tubing is then rotated and moved longitudinally relative to the laser which is also machine-controlled. The laser selectively removes the material from the tubing by ablation and a pattern is cut into the tube. The tube is therefore cut into the discrete pattern of the finished stent. As mentioned above, the automated laser device could also remove an amount of material from the surface of the stress relievers to create a stress reliever having a wall thickness that is less than the wall thickness of the struts forming the stent. Further details on how the tubing can be cut by a laser are found in U.S. Pat. Nos. 5,759,192 (Saunders), 5,780,807 (Saunders) and 6,131,266 (Saunders), which are incorporated herein in their entirety.

The process of cutting a pattern for the stent into the tubing generally is automated except for loading and unloading the length of tubing. For example, a pattern can be cut in tubing using a CNC-opposing collet fixture for axial rotation of the length of tubing, in conjunction with CNC X/Y table to move the length of tubing axially relative to a machine-controlled laser as described. The entire space between collets can be patterned using the CO2 or Nd:YAG laser set-up. The program for control of the apparatus is dependent on the particular configuration used and the pattern to be ablated in the coding.

After the stent has been cut by the laser, electrical chemical polishing, using various techniques known in the art, should be employed in order to create the desired final polished finish for the stent. The electropolishing will also be able to take off protruding edges and rough surfaces which were created during the laser cutting procedure.

Any of the stents and stress relievers disclosed herein can be coated with a drug for treating the vascular system. The drug, therapeutic substance or active agent, terms which are used interchangeably, in the coating can inhibit the activity of vascular smooth muscle cells. More specifically, the active agent can be aimed at inhibiting abnormal or inappropriate migration and/or proliferation of smooth muscle cells for the inhibition of restenosis. The active agent can also include any substance capable of exerting a therapeutic or prophylactic effect for a diseased condition. For example, the agent can be for enhancing wound healing in a vascular site or improving the structural and elastic properties of the vascular site. Examples of agents include antiproliferative substances such as actinomycin D, or derivatives and analogs thereof (manufactured by Sigma-Aldrich, Inc., Milwaukee, Wis.; or COSMEGEN available from Merck & Co., Inc., Whitehorse Station, N.J.). Synonyms of actinomycin D include dactinomycin, actinomycin IV, actinomycin I1, actinomycin X1, and actinomycin C1. The active agent can also fall under the genus of antineoplastic, anti-inflammatory, antiplatelet, anticoagulant, antifibrin, antithrombin, antimitotic, antibiotic, antiallergic and antioxidant substances. Examples of such antineoplastics and/or antimitotics include paclitaxel (e.g., TAXOL® by Bristol-Myers Squibb Co., Stamford, Conn.), docetaxel (e.g., Taxotere®, from Aventis S. A., Frankfurt, Germany), methotrexate, azathioprine, vincristine, vinblastine, fluorouracil, doxorubicin hydrochloride (e.g., Adriamycin® from Pharmacia & Upjohn, Peapack, N.J.), and mitomycin (e.g., Mutamycin® from Bristol-Myers Squibb Co.). Examples of such antiplatelets, anticoagulants, antifibrin, and antithrombins include sodium heparin, low molecular weight heparins, heparinoids, hirudin, argatroban, forskolin, vapiprost, pro-arg-chloromethylketone (synthetic antithrombin), dipyridamole, flycoprotein IIb/IIIa platelet membrane receptor antagonist antibody, recombinant hirudin, and thrombin inhibitors such as Angiomax ä (Biogen, Inc., Cambridge, Mass.). Examples of such cytostatic or antiproliferative agents include angiopeptin, angiotensin converting enzyme inhibitors such as captopril (e.g., Capoten® and Capozide® from Bristol-Myers Squibb Co.), cilazapril or lisinopril (e.g., Prinvil® and Prinzide® from Merck & Co., Inc.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), and nitric oxide. An example of an antiallergic agent is permirolast potassium. Other therapeutic substances or agents which may be appropriate include alpha-interferon, genetically engineered epithelial cells, rapamycin and it derivatives and analogs, and dexamethasone.

Coating can be made from any suitable biocompatible polymer, examples of which include ethylene vinyl alcohol copolymer (commonly known by the generic name EVOH or by the trade name EVAL); poly(hydroxyvalerate); poly (L-lactic acid); polycaprolactone; poly(lactide-co-gly-colide); poly(hydroxybutyrate); poly(hydroxybutyrate-co-valerate); polydioxanone; polyorthoester; polyanhydride; poly(glycolic acid); poly(D,L-lactic acid); poly(flycolic acid-co-trimethylene carbonate); polyphosphoester; poly-phosphoester urethane; poly(aminoacids); cyanoacrylates; poly(trimethylene carbonate); poly(iminocarbonate); copoly(ether-esters) (e.g., PEO/PLA); polyalkylene oxalates; poly-phosphazenes; biomolecules, such as fibrin, fibrinogen, cellulose, starch, collagen and hyaluronic acid; polyurethanes; silicones; polyesters; polyolefin often intricate strut patterns which form the stent structure ns; polyisobutylene and ethylene-alphaolefin copolymers; acrylic polymers and copolymers; vinyl halide polymers and copolymers, such as polyvinyl chloride; polyvinyl ethers, such as polyvinyl methyl ether; polyvinylidene halides, such as polyvinylidene fluoride and polyvinylidene chloride; polyacrylonitrile; polyvinyl ketones, polyvinyl aromatics, such as polystyrene; polyvinyl esters, such as polyvinyl acetate; copolymers of vinyl monomers with each other and olefins, such as ethylenemethyl methacrylate copolymers, acrylonitrile-styrene copolymers, ABS resins, and ethylene-vinyl acetate copolymers; polyamides, such as Nylon 66 and polycaprolactam; alkyd resins; polycarbonates; polyoxymethylenes; polyimides; polyethers; epoxy resins; polyurethanes; rayon; rayon-triacetate; cellulose; cellulose acetate; cellulose butyrate; cellulose acetate butyrate; cellophane; cellulose nitrate; cellulose propionate; cellulose ethers; and carboxymethyl cellulose. Coating 20 can also be silicon foam, neoprene, santoprene, or closed cell foam.

Although the present invention has been described in terms of certain preferred embodiments, other embodiments that are apparent to those of ordinary skill in the art are also within the scope of the invention. Accordingly, the scope of the invention is intended to be defined only by reference to the appended claims. While the dimensions, types of materials and coatings described herein are intended to define the parameters of the invention, they are by no means limiting and are exemplary embodiments.

I claim:

1. A stent, comprising:
a series of strut pairs, each strut pair formed by two adjacent struts connected together at a strut junction, the series of strut pairs collectively forming a generally tubular stent body having a first delivery diameter and a second implanted diameter; and
at least one stress reliever associated with each strut pair, each stress reliever reducing the amount of strain experienced by the strut pair when the stent body moves between the first delivery diameter and the second implanted diameter.

2. The stent of claim 1, wherein each stress reliever is located at the strut junction of each strut pair and is disposed between the adjacent struts forming the strut pair.

3. The stent of claim 1, wherein some of the stress relievers are in tension as the strut pairs move into the second implanted diameter.

4. The stent of claim 1, wherein some of the stress relievers are in compression as the strut pairs move into the second implanted diameter.

5. The stent of claim 1, wherein some of the pairs of struts include two stress relievers associated with the strut pair.

6. The stent of claim 1, further including a series of strut groups, each strut group made from three or more struts connected together at a strut junction with at least one stress reliever associated with each strut group.

7. The stent of claim 6, wherein some of the strut groups are formed from four struts connected together at the strut junction to form an X-shaped pattern when the stent body is in the second implanted diameter and at least one stress reliever is disposed between adjacent struts forming the X-shaped pattern of the strut group.

8. The stent of claim 7, wherein some of the strut group have at least two stress relievers disposed between adjacent struts forming the strut group.

9. The stent of claim 1, wherein some of the strut pairs are connected together at the same strut junction.

10. The stent of claim 1, wherein each adjacent strut forming the strut pair includes a first end and a second end, the first ends of each adjacent strut being connected together at a strut junction and the second ends of each adjacent strut being attached to an adjacent strut junction.

11. The stent of claim 10, wherein all of the first ends and second ends of the struts forming the strut pairs are attached to a strut junction.

12. A stent, comprising:
a series of strut groups collectively forming a generally tubular stent body having a first delivery diameter and a second implanted diameter, each strut group including at least two adjacent struts connected together at a strut junction; and
at least one stress reliever associated with each strut group, each stress reliever reducing the amount of strain experienced by the struts forming the strut group when the stent body moves between the first delivery diameter and the second implanted diameter.

13. The stent of claim 12, wherein some of the strut groups have two struts joined together to form the strut group.

14. The stent of claim 13, wherein some of the strut groups have two struts joined together to form the strut group.

15. The stent of claim 12, wherein at least one connecting link attaches each elongate strut member to an adjacent elongate strut member so that at least a portion of the connecting link is positioned within the peak as it attaches that peak to a peak of an adjacent elongate strut member.

16. The stent of claim 12 wherein the peaks and valleys of each elongate strut member are in phase with the peaks and valleys of adjacent elongate members.

17. The stent of claim 12, further including:
a second series of strut groups collectively forming a second, generally tubular stent body having a first delivery diameter and a second implanted diameter, each strut group including at least two adjacent struts connected together at a strut junction; and
at least one connecting link connecting the first-mention stent body to the second generally tubular stent body.

18. A stent, comprising:
a series of strut groups collectively forming a generally tubular stent body having a first delivery diameter and a second implanted diameter, each strut group including at least two adjacent struts connected together at a strut junction; and
at least one stress reliever associated with each strut group, each stress reliever reducing the amount of strain experienced by the struts forming the strut group when the stent body moves between the first delivery diameter and the second implanted diameter.

19. The stent of claim 17, wherein a plurality of connecting links connect each elongate strut member to an adjacent strut member, some of the connecting links being disposed laterally adjacent to each other along the tubular stent body to form a first set of connecting links and some of the connecting links being disposed laterally adjacent to each other along the tubular stent body to form a second set of connecting links.

20. The stent of claim 17, wherein each of the connecting links have a first end attached to the peak of an elongate strut member and a second end attached to the peak of an adjacent elongate strut member.
